(19) United States
(12) Patent Application Publication
(10) Pub. No.: US 2006/0025688 A1
(43) Pub. Date: Feb. 2, 2006

(54) Blood flow visualizing diagnostic apparatus

(75) Inventors: Toshiyuki Hayase, Sendai-shi (JP); Kenichi Funamoto, Sendai-shi (JP); Atsushi Shirai, Sendai-shi (JP); Tomoyuki Yambe, Sendai-shi (JP); Yoshifumi Saijo, Sendai-shi (JP)

Correspondence Address: Norman P Soloway;Hayes Soloway, 130 W Cushing Street, Tucson, AZ, 85701, US

(73) Assignee: TOHOKU TECHNO ARCH CO. LTD., Miyagi (JP)

(21) Appl. No.: 10/527,140

(22) PCT Filed: Oct. 2, 2003
(86) PCT No.: PCT/JP03/12689

§ 371 Date: Oct. 19, 2005

(30) Foreign Application Priority Data
Oct. 7, 2002 (JP) 2002-293631

(51) Int. Cl. A61B 8/00

(52) U.S. Cl. 600/454; 600/438

(57) ABSTRACT

A blood flow visualizing diagnostic apparatus characterized by having an ultrasonic measurement unit 120 which emits an ultrasonic beam toward a blood vessel inside a human body to receive the reflected ultrasonic signal, an analysis processing unit 220 which obtains a blood vessel shape and a blood flow velocity in the blood vessel by the received signal, a simulation unit 244 which sets computational lattices on the basis of the blood vessel shape obtained by the analysis processing unit 220 to simulate the blood flow velocity and a pressure distribution, a feedback unit 246 which computes an error between the blood flow velocity obtained by the analysis processing unit and the blood flow velocity obtained by the simulation unit 244 to feed back the error to the simulation unit 244, and display units 260 and 140 which display the blood flow velocity and the pressure distribution output from the simulation unit after the feedback.

### TECHNICAL FIELD

The present invention relates to ultrasonic measurement of blood which flows through a blood vessel, particularly to measurement of a blood flow velocity and a pressure distribution.

### BACKGROUND ART

Conventionally, an ultrasonic Doppler diagnostic apparatus is used as a method to know the blood flow. The ultrasonic Doppler diagnostic apparatus is one in which a velocity component of the blood flow parallel to the ultrasound been emitted from a probe is detected by Doppler effect to display the velocity vector approaching to the probe or coming away from the probe in color. However, because usually the ultrasonic probe comes into vertical contact with a human skin, the velocity component of the blood flow parallel to the ultrasound been emitted from the probe is small in almost all of the blood vessels running in parallel with the human skin. Therefore, it is difficult to correctly display the velocity of the blood flow. As described above, as only one specific directional component can be measured in three directional components of the velocity vector of the blood flow, the blood flow cannot be accurately displayed in the conventional ultrasonic Doppler diagnostic apparatus (for example, see Patent documents 1 and 2). Currently, there is no technology for measuring the pressure distribution in the blood vessel, which is important to prediction of rupture of the disabled blood vessel.

In order to obtain detailed information of blood flow in the blood vessel, it is thought that numerical simulation is effective. However, in the cases where a bifurcation, a curvature, an ulcer or a stricture, exists in the blood vessel, it is difficult to determine a boundary condition, and, therefore, sufficient computational accuracy is not obtained.

In conventional numerical simulations, a SIMPLER method is well known as a simulation method of a flow field (for example, see Non-Patent Document 1).

The SIMPLER method is briefly described below referring to a flowchart shown in FIG. 1 (for example, see Non-Patent document 1 for more detailed information).

A Navier-Stokes equation and a continuity equation are generally expressed by the following equations.

[Equation 1]
∂u/∂=f(u,p) (1)
divu=0 (2)

The equation (1) is one in which three generalized conservation laws of the momentus for the three components (u, v, w) of the velocity vector u are expressed as a whole. In the equations (1) and (2), it is assumed that density p is constant in the whole flow field.

The continuity equation (2) is expressed by the following equation when a Cartesian coordinate is used.

[Equation 2]
∂u/∂x+∂v/∂y+∂w/∂z=0 (3)

When the equation (3) is integrated by a control volume whose center is a lattice point, the following equation is obtained.

[Equation 3]
(u_{E}−u_{w})ΔyΔz+(v_{N}−v_{S})ΔxΔz+(w_{D}−w_{U})ΔxΔz (4)

The following equation is obtained from a discrete form of the Navier-Stokes equation for the velocity u.

[Equation 4]
u_{w}=(ΣBⱼuⱼ+S_{w})/B_{w}+d_{w}(pₒ−p_{w}) (5)
In the case of a three dimension, (ΣBⱼuⱼ) in the equation (5) represents a sum of six values around u_{w}. A first term in a right-hand side of the equation (5) is set as follows:
[Equation 5]
û_{w}=(ΣBⱼuⱼ+S_{w})/B_{w} (6)
When the equation (5) is substituted for the equation (4), the following equation (7) of the generalized conservation law is obtained for the pressure.
[Equation 6]
a₀p₀=a_{E}p_{E}+a_{w}p_{w}+a_{N}p_{N}+a_{S}p_{S}+a_{D}p_{D}+a_{U}p_{U}+S_{O}(û_{w}, . . . ) (7)
The equation (7) is referred to as a pressure equation. The velocities u, v, and w and the pressure p which simultaneously satisfy the momentum equation (5) and the pressure equation (7) are determined by an iterative method. In order to stabilize the computation, a correction is performed in each step of the iteration so that a velocity field satisfies the continuity equation. Namely, when solutions of the momentum equation for a pressure field p* including the error is set to u_{w}* and the like, the solutions do not generally satisfy the continuity equation. Assuming that true solutions are u (vector) and p, u (vector) and p are expressed as follows using a correction term u′ (vector) and p′.
[Equation 7]
p=p*+p′
u=u*+u′ (8)
The above equation (8) is substituted for the equation (5) and the effect of the amount of surrounding velocity correction uⱼ′ is neglected. Consequently, the following equation is obtained.
[Equation 8]
u′_{w}=(p′ₒ−p′_{w})d_{w} (9)
When the equation (9) is substituted for the equation (8), the following velocity correction equation is obtained.
[Equation 9]
u_{w}=u*_{w}+(p′ₒ−p′_{w})d_{w} (10)
Further, when the equation (10) is substituted for the equation (4), a discrete equation for the amount of pressure correction is obtained as follows:
[Equation 10]
a₀p′₀=a_{E}p′_{E}+a_{w}p′_{w}+a_{N}p′_{N}+a_{S}p′_{S}+a_{D}p′_{D}+a_{U}p′_{U}+S_{O}(u*_{w}, . . . ) (11)

In summary, the numerical analysis technique referred to as the SIMPLER method is obtained.

FIG. 1 shows the flowchart of a computational procedure by the SIMPLER method. In the flowchart of FIG. 1, the velocity field is fixed first, and û_{w} and the like are computed in each lattice point from the equation (6) (S102). The pressure field p is determined from the pressure equation (7) using the obtained values for û_{w} etc. (S104). The velocity field is determined from the Navier-Stokes equation (5) (S106). The velocity is corrected by the pressure correction equation (11) and the velocity correction equation (10) (S108), and then checked to decide whether the computation converges or not (S110). The solution is obtained for a time step n by repeating the computational procedure from S102 to S110 until the computation converges.

In order to reproduce the actual blood flow by using the above-described numerical simulation of the flow field, it is necessary to give a complete state (initial condition) of the blood flow at a certain time and a state in a boundary surface (boundary condition) through all the times. However, it is realistically impossible to give the exact initial condition and the boundary condition.

There are Non-Patent Documents 2 to 7 in which measurement data of the actual flow field is fed back to the numerical analysis method (numerical simulation). In the Non-Patent Documents 2 and 3, a turbulent flow field in a square duct is analyzed. In the Non-Patent Documents 4 to 7, a Karman vortex in a wake flow of a prism placed in a square channel is analyzed. In the Non-Patent Documents 2 and 3, the error is partially decreased by performing the feedback to the pressure boundary condition from the error in the velocity at a certain position in the flow direction. In the Non-Patent Documents 4 to 7, the feedback is performed to the pressure at few points on a prism from the error in the pressure. However, there is no description concerning the application of the simulation to the actual blood flow. Further, it is not described that the whole error is uniformly decreased when sufficient number of points are distributed over the flow direction to perform the feedback with respect to the velocity.

[Patent Document 1]

Japanese Patent Laid-Open Publication No.2000-229078
   [Patent Document 2]
Japanese Patent Laid-Open Publication No.2001-218768
   [Non-Patent Document 1]
Hayase: Finite volume method (SIMPLER method), Journal of the Japan Hydraulics & Pneumatics Society (in Japanese), Vol. 26, No. 4(1995), pp. 407-413.
   [Non-Patent Document 2]
Hayase and Hayashi: Fundamental Study on Computer-Aided Flow Field Control (State Observer for Flow System), Transactions of the Japan Society of Mechanical Engineers (in Japanese), Vol. 62, No. 598(1996), pp. 2261-2268.
   [Non-Patent Document 3]
Hayase, T., and Hayashi, S.: State Estimator of Flow as an Integrated Computational Method with the Feedback of Online Experimental Measurement, Transactions of the ASME, J. Fluids Eng., Vol. 119(1997), pp. 814-822.
   [Non-Patent Document 4]
Nisugi, Takeda, Shirai, and Hayase: Fundamental Study on Hybrid Wind Tunnel (Study of Feedback Scheme), Proceedings of the JSME Fluids Engineering Division Meeting (in Japanese), CD-ROM (2001), G803.
   [Non-Patent Document 5]
Takeda, Nisugi, Shirai, and Hayase: Fundamental Study on Hybrid Wind Tunnel (Evaluation of Estimation Performance), Proceedings of the JSME Fluids Engineering Division Meeting (in Japanese), CD-ROM (2001), G804.
   [Non-Patent Document 6]
Hayase, T., Nisugi, K. and Shirai, A.: Numerical Realization of Flow Field by Integrating Computation and Measurement, Proceedings of 5^{th} World Congress on Computational Mechanics, Vienna, Austria, Jul. 7-12 (2002).
   [Non-Patent Document 7]
Hayase Toshiyuki: “Numerical simulation and Virtual Measurement for flow Fields” Measurement and Control, Vol 40, No. 11 (November 2001), pp. 790-794.

An object of the invention is to provide a diagnostic apparatus which can display the pressure distribution of the blood while accurately displaying the blood flow velocity distribution in the blood vessel.

### DISCLOSURE OF THE INVENTION

In order to achieve the object, the invention is a blood flow visualizing diagnostic apparatus characterized by having an ultrasonic measurement unit which emits an ultrasonic signal toward a blood vessel inside a human body to receive the reflected ultrasonic signal, an analysis processing unit which obtains a blood vessel shape and a blood flow velocity in the blood vessel by the received signal, a simulation unit which sets computational lattices on the basis of the blood vessel shape obtained by the analysis processing unit to simulate the blood flow velocity vector distribution and the pressure distribution, a feedback unit which computes an error between the blood flow velocity obtained by the analysis processing unit and the blood flow velocity obtained by the simulation unit to feed back the error to the simulation unit, and a display unit which displays the blood flow velocity distribution and the pressure distribution output from the simulation unit after the feedback.

It is desirable that the feedback unit performs the feedback to representative points which are distributed over the flow domain in the computational lattices.

### BRIEF DESCRIPTION OF THE DRAWINGS

FIG. 1 is a flowchart of the conventional numerical simulation (SIMPLER method);

FIG. 2 is a block diagram showing a configuration of an embodiment of the invention;

FIG. 3 is a view showing a display example of color Doppler image of blood flow;

FIG. 4 is a view showing an example of computational lattices used for simulation;

FIG. 5 is a view showing an example of a velocity boundary condition given to the simulation;

FIG. 6 is a view showing an example of representative points for performing feedback;

FIG. 7 is a view for explaining the feedback with respect to the representative point;

FIG. 8 is a flowchart of the simulation by the feedback;

FIG. 9A is a view showing simulation result by the feedback;

FIG. 9B is a view showing simulation result by the feedback;

FIG. 10A is a view showing comparison between measurement integrated simulation and the conventional simulation; and

FIG. 10B is a view showing comparison between measurement integrated simulation and the conventional simulation.

### BEST MODE FOR CARRYING OUT THE INVENTION

Referring to the accompanying drawings, an embodiment of the invention will be described below.

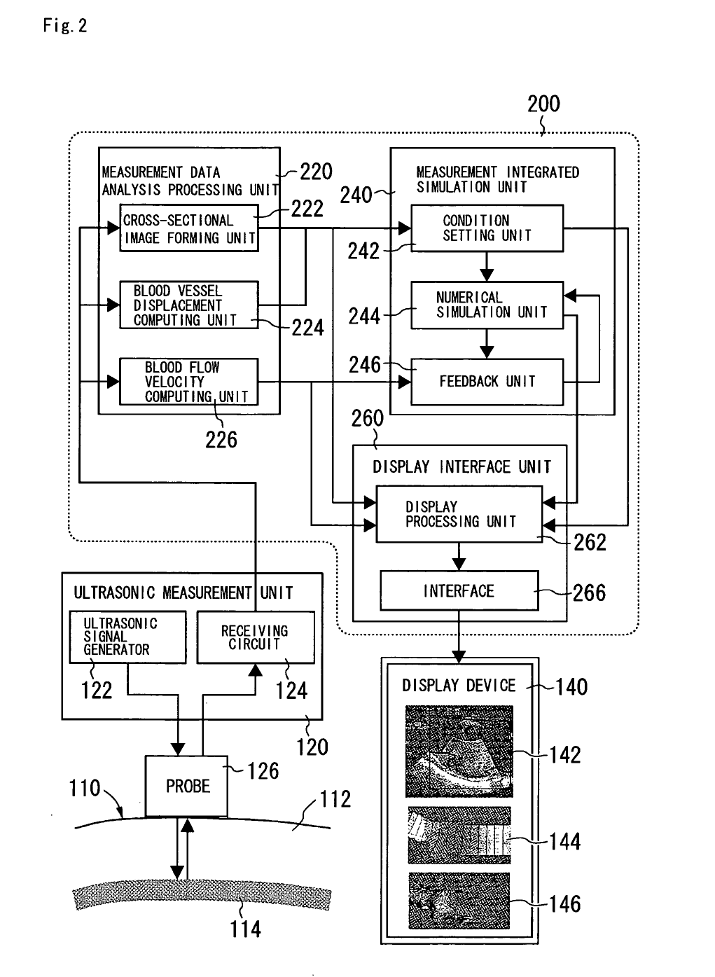

FIG. 2 shows a block diagram of an overall configuration of a blood flow visualizing diagnostic apparatus according to the invention using the ultrasonic measurement integrated simulation.

In FIG. 2, in an ultrasonic measurement unit 120, an ultrasonic signal generator 122 generates a signal to transmit an ultrasonic pulse from a probe 126 which is in contact with a skin 112 of a human 110. The transmitted ultrasonic pulse is reflected from a blood vessel 114 and the like to become an echo signal. A receiving circuit 124 amplifies and processes the echo signal through the probe 126 to transmit the echo signal to a measurement data analysis processing unit 220 in a measurement data processing unit 200. The ultrasonic pulse is transmitted from the probe 126 so that an image in a certain range is formed by, e.g. performing electronic scan.

The measurement data analysis processing unit 220 includes a cross-sectional image forming unit 222 which forms a cross-sectional image from the echo signal, a blood vessel displacement computing unit 224 which computes displacement of the blood vessel, and a blood flow velocity computing unit 226 which utilizes the Doppler effect to compute the blood flow velocity in the blood vessel. The measurement data analysis processing unit 220 computes the result of the ultrasonic measurement. The measurement results are displayed on a display device 140 through an interface 266 while color-coded according to, e.g. the velocity by a display processing unit 262 in a display interface unit 260.

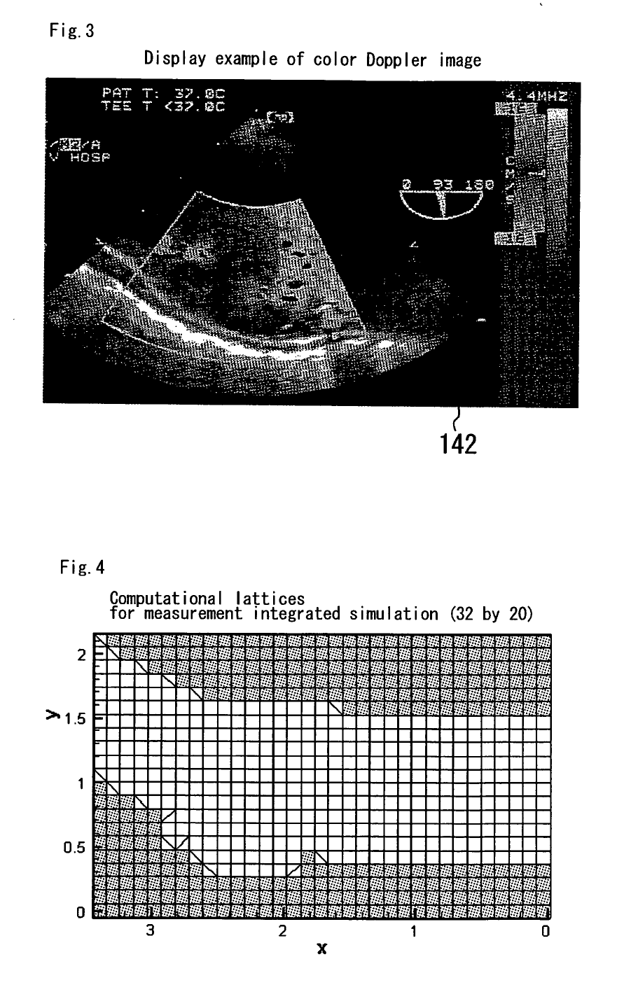

FIG. 3 shows an example of the conventional color Doppler image output by the display processing unit 262 shown in FIG. 2. The display of the image includes a cross-sectional blood vessel image generated by the cross-sectional image forming unit 222 and a blood flow velocity component in the ultrasonic beam direction generated by the blood flow velocity computing unit 226 (for example, see Patent Documents 1 and 2 and the like) The blood flow visualizing diagnostic apparatus according to the invention has a function of computing the blood flow velocity and the pressure distribution in the blood vessel or a heart by the ultrasonic measurement integrated simulation (measurement integrated simulation unit 240). The measurement integrated simulation unit 240 includes a condition setting unit 242 which generates computational lattices by performing binarization of the cross-sectional blood vessel image from the cross-sectional image forming unit 222 and the blood vessel displacement computing unit 224, a numerical simulation unit 244 which performs numerical simulation by using the computational lattice generated by the condition setting unit 242, and a feedback unit 246 which computes the amount of feedback of the blood flow velocity by the measurement data to perform the feedback to numerical simulation unit 244. For the reference purpose, the blood flow simulation executed by the numerical simulation unit 244 is described in, e.g. Non-Patent Documents 1 and 2. The velocity and the pressure of the blood flow at each lattice point can be determined in the numerical simulation described in the documents.

Then, the measurement integrated simulation unit 240 will be described in detail.

FIG. 4 shows the blood vessel shape and computational lattices, which are obtained by the condition setting unit 242 in the measurement integrated simulation unit. The condition setting unit 242 generates the computational lattices used for the numerical analysis of the flow while performing the binarization of the cross-sectional blood flow image generated by the cross-sectional image forming unit 222. A velocity vector and pressure of the blood flow in the generated blood vessel shape and lattice points (point at which a vertical line and a horizontal line intersect each other) are evaluated by the numerical computing of the flow executed in the numerical simulation unit 244 mentioned later.

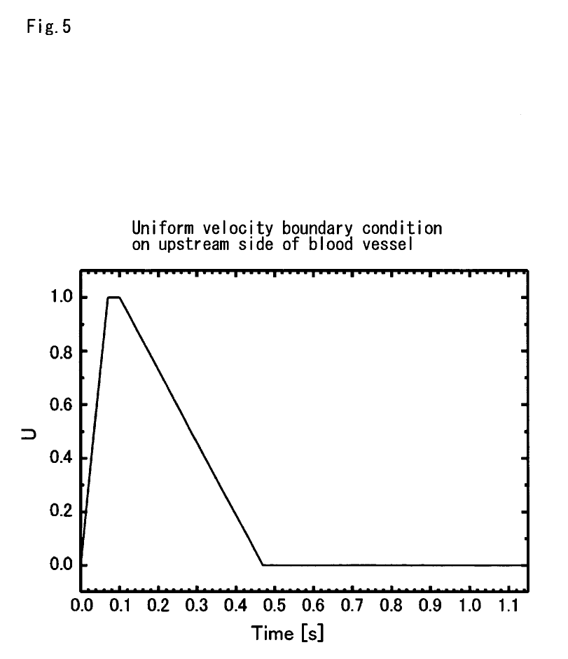

In the numerical simulation of the flow in the ultrasonic measurement integrated simulation, it is necessary to give a boundary condition of the velocity or the pressure in a boundary of a target domain. FIG. 5 shows modeling of time change in the blood flow velocity in the center of the cross section obtained by the ultrasonic measurement. Assuming that the blood flow is the uniform flow in parallel with a blood vessel wall at the upstream cross section, the time change in the blood flow is given in FIG. 5. However, as the assumption that the blood flow is the uniform flow in parallel with a blood vessel wall is not always valid in the actual blood flow, an error due to the inappropriate boundary condition cannot be avoided. On the contrary, in the ultrasonic measurement integrated simulation, the error can be cancelled by the feedback of the measurement data.

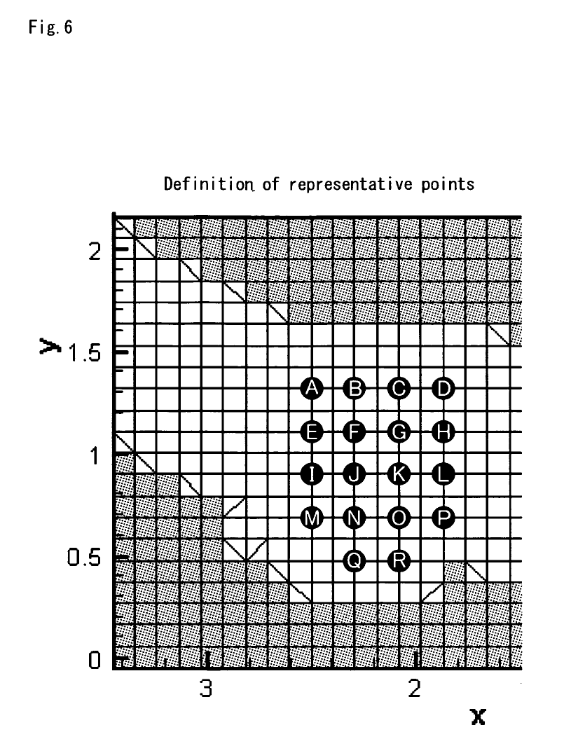

FIG. 6 is a view showing representative points in the measurement integrated simulation (18 points of A to R in FIG. 6). The feedback unit 246 determines an error between the blood flow velocity obtained by the ultrasonic measurement and the corresponding result of the numerical simulation, and causes the result of the numerical simulation to converge to a value of the actual blood flow by feeding back body force according to the error to the numerical simulation.

In the SIMPLER method, the feedback is performed by adding body force f (vector) to an end of a right-hand side in the equation (5) of the momentum conservation equation which is of the Navier-Stokes equation.

[Equation 11]
u_{W}=(ΣBⱼuⱼ+S_{W})/B_{W}+d_{W}(p_{O}−p_{W})+f_{W} (5)′

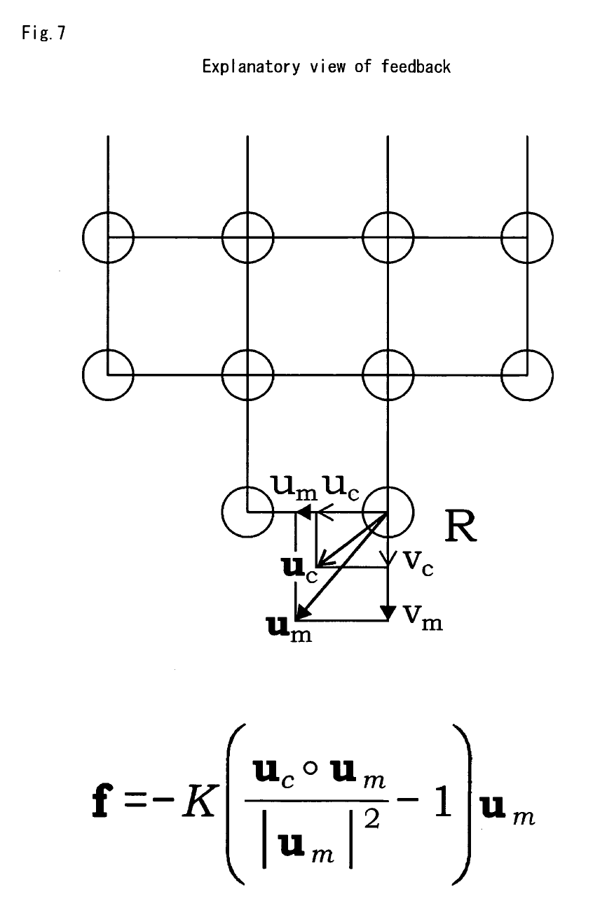

FIG. 7 is a view for explaining the feedback at the representative point performed in the numerical simulation unit 244. In this case, the point R will be described as an example of the representative points. When the numerical simulation and the measurement are simultaneously performed, the velocity vector obtained by the numerical simulation is set to u_{c}, and expressed in a two-dimensional way. A difference between a component in the ultrasonic beam direction of the velocity vector u_{c} obtained by the Navier-Stokes equation which is of the momentum conservation equation and a corresponding velocity component of the velocity vector uₘ (vector) in the ultrasonic beam direction obtained by the measurement is fed back to the body force term in the Navier-Stokes equation.

The term of the body force f (vector) used in the actual feedback is expressed by the following equation:
f=·K{(u_{c}ouₘ/|uₘ|²)−1}uₘ [Equation 12]
where the vector u_{c} is [uₒ, v_{c}, w_{c}], the vector uₘ is [uₘ, vₘ, wₘ, and K is a gain of the feedback. The body force vector f determined by the above equation is given to a sufficiently large number of representative points distributed over the computing domain.

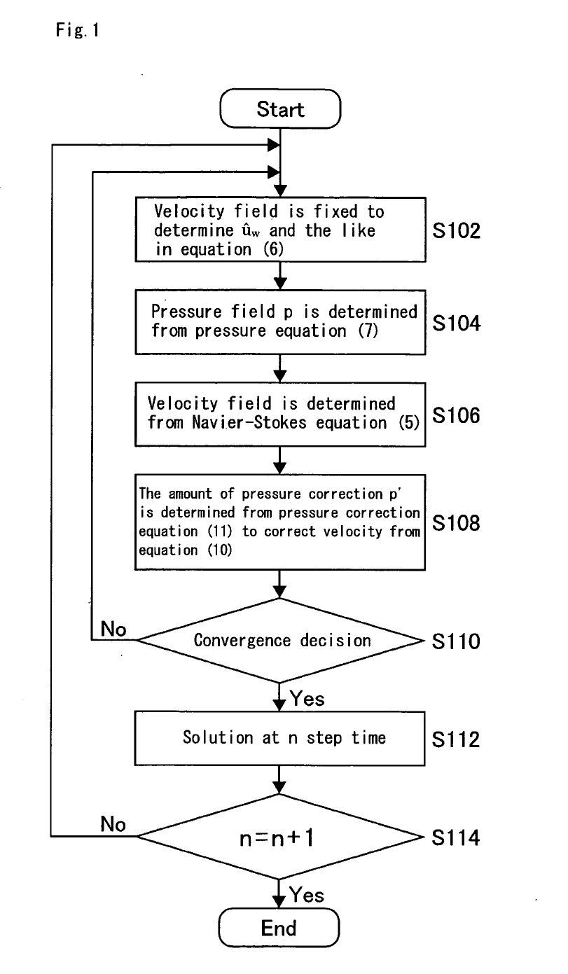

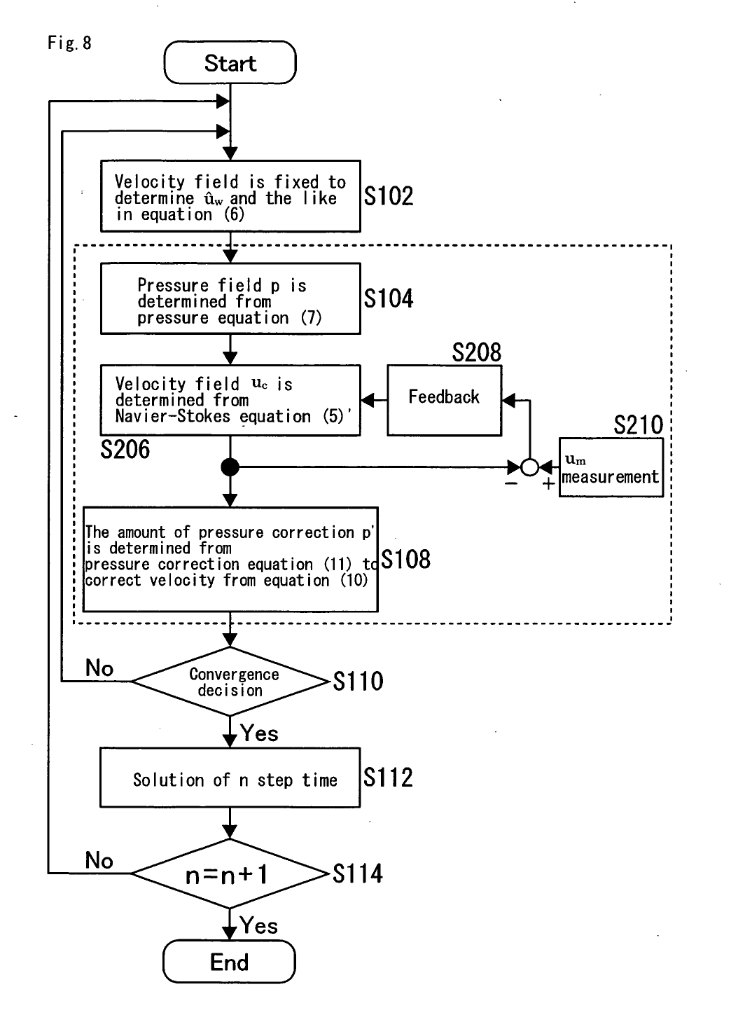

FIG. 8 is a flowchart for explaining the feedback when the SIMPLER method is used as the numerical simulation in the embodiment. It is also possible to use another numerical simulation. The same processing as the flowchart of FIG. 1 is performed in the step indicated by the same sign as the flowchart of FIG. 1.

In FIG. 8, the measurement result urn (vector) is obtained from the measurement data analysis processing unit 220 (S210), and the body force is determined in order to perform the feedback (S208). Then, as described above, the computation is performed by adding the computed body force to the Navier-Stokes equation at each representative point (S206). In other steps, the same processing shown in FIG. 1 is performed.

Thus, in the ultrasonic measurement integrated simulation, the body force f (vector) having the magnitude proportional to the difference between the ultrasonic measurement result and the corresponding simulation result is fed back to the momentum conservation equation in the numerical simulation. The beam direction component of the computed velocity u_{c} (vector) in the numerical simulation is brought asymptotically close to that of the corresponding measurement velocity uₘ (vector).

The feedback rule described above holds for an arbitrary velocity direction obtained by the ultrasonic measurement.

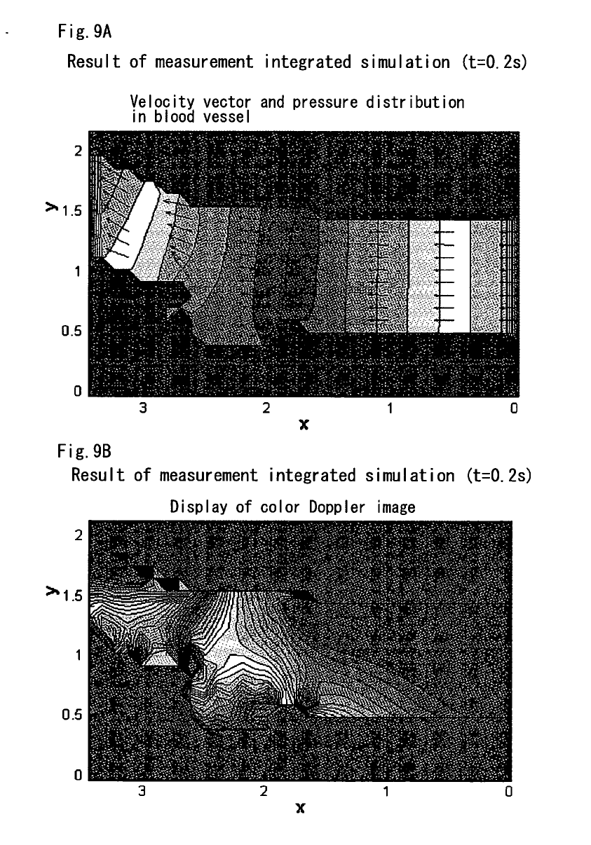

FIG. 9A,B shows the result of the ultrasonic measurement integrated simulation. FIG. 9A shows the pressure distribution in the cross section of the blood vessel and the velocity vector of the blood flow. Although only a part of the velocity vectors is shown in FIG. 9A for illustrative purposes, the velocity vectors and the pressures are actually obtained at all the lattice points shown in FIG. 4. FIG. 9B shows the display of a color Doppler image by using information on the velocity obtained by the ultrasonic measurement integrated simulation.

The result of a comparison between the ultrasonic measurement integrated simulation and the conventional numerical simulation is shown below for the computational accuracy.

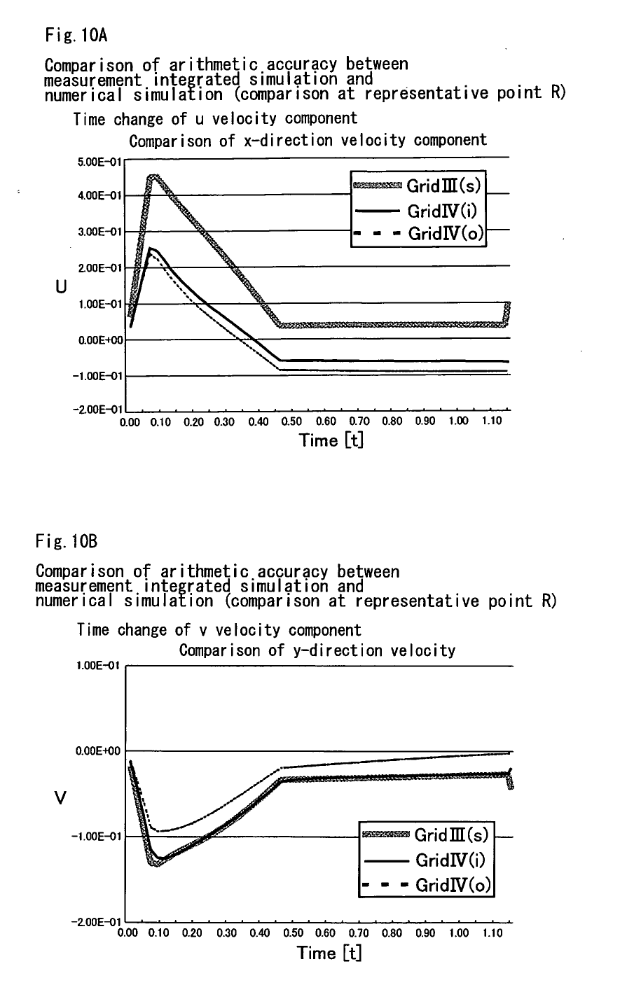

FIG. 10A,B shows time changes in the velocity components u and v in the x and y-directions of the blood flow at the representative point R shown in FIG. 6. In order to precisely evaluate the computational accuracy, the numerical simulation was performed using the computational lattices in which the number of lattice points of the computational lattice shown in FIG. 4 is doubled in the x and y-directions. Then, the evaluation of the accuracy was made on the basis of the result. In FIG. 10A,B, a solid line represents the velocity change which becomes a standard. A thin line of FIG. 10A,B represents the result in which the feedback was performed by the method shown in FIG. 7 with the y-direction velocity components v of the representative points A to R in the standard flow field. A dot line of FIG. 10A,B represents the result in which the coarse lattice system shown in FIG. 4 was used to perform the conventional numerical simulation without performing the feedback. In the conventional numerical simulation, the results of the velocity components u and v differ from the results of the standard solution respectively. The difference is caused by the insufficient lattice spacing of the computational lattice. On the contrary, in the results of the measurement integrated simulation in which the feedback was performed, since the error in the y-direction was fed back to the measurement integrated simulation, the result substantially equal to the standard solution is obtained for the y-direction velocity v, and the result close to the standard solution compared with the conventional simulation is obtained for the x-direction velocity u.

Table 1 shows a comparison of the accuracy of the numerical solution by the measurement integrated simulation. The accuracy was evaluated with an error norm which is a mean value of the whole in which absolute values of difference between the standard solution of the y-direction velocity v and the computational result are averaged out by time.

TABLE 1

| | Error norm |
|---|---|
| Measurement integrated simulation | 0.0025 |
| Conventional numerical simulation | 0.0202 |

As can be seen from Table 1, when compared with the conventional numerical simulation, the error is decreased by about one digit.

### INDUSTRIAL APPLICABILITY

Since the blood flow velocity in the blood vessel and the pressure distribution can be accurately displayed using the diagnostic apparatus according to the invention, the diagnostic apparatus according to the invention can be used for the accurate diagnosis and a therapeutic plan for physical-shape pathologic changes inside the blood vessel such as aortic stricture or ulcer.

1. A blood flow visualizing diagnostic apparatus characterized by having:
an ultrasonic measurement unit which emits an ultrasonic signal toward a blood vessel inside a human body to receive the reflected ultrasonic signal;
an analysis processing unit which obtains a blood vessel shape and a blood flow velocity in the blood vessel by the received signal;
a simulation unit which sets computational lattices on the basis of the blood vessel shape obtained by said analysis processing unit to simulate the blood flow velocity and a pressure distribution;
a feedback unit which computes an error between the blood flow velocity obtained by said analysis processing unit and the blood flow velocity obtained by said simulation unit to feed back the error to said simulation unit; and
a display unit which displays the blood flow velocity and the pressure distribution output from said simulation unit after the feedback.

2. A blood flow visualizing diagnostic apparatus according to claim 1, characterized in that said feedback unit performs the feedback to a sufficiently large number of representative points which are distributed over the blood flow domain in said computational lattices.
